(19) United States
(12) Patent Application Publication
(10) Pub. No.: US 2026/0294845 A1
(43) Pub. Date: Oct. 1, 2026

(54) Uses of Adrenalone to Manage Cardiotoxicity or Cardiovascular Disorders

(71) Applicants: Emory University, Atlanta, GA (US); Children's Healthcare of Atlanta, Inc., Atlanta, GA (US)

(72) Inventors: Chunhui Xu, Decatur, GA (US); Antonio Rampoldi, Atlanta, GA (US); Yuhong Du, Atlanta, GA (US)

(21) Appl. No.: 19/481,130

(22) PCT Filed: May 3, 2024
(86) PCT No.: PCT/US2024/027821

§ 371 Date: Nov. 3, 2025

Related U.S. Application Data
(60) Provisional application No. 63/463,726, filed on May 3, 2023.

(51) Int. Cl. A61K 31/137; A61K 31/496; A61P 9/00; A61P 39/00; A61P 39/06

(52) U.S. Cl. CPC A61K 31/137; A61K 31/496; A61P 9/00; A61P 39/00; A61P 39/06

(57) ABSTRACT

This disclosure relates to methods of treating or preventing cardiotoxicity or a cardiac disease or condition by administering adrenalone, derivative, prodrug, ester, or salt thereof to a subject in need thereof. In certain embodiments, the subject is in need thereof due to administration of or consumption of addictive substances or therapeutic agents that poses a risk of cardiotoxicity.

### CROSS-REFERENCE TO RELATED APPLICATIONS

This application claims the benefit of U.S. Provisional Application No. 63/463,726 filed May 3, 2023. The entirety of this application is hereby incorporated by reference for all purposes.

### STATEMENT REGARDING FEDERALLY SPONSORED RESEARCH OR DEVELOPMENT

This invention was made with government support under AA028527 and HL136345 awarded by the National Institutes of Health. The government has certain rights in the invention.

### BACKGROUND

Alcohol addiction is common. Aside from the liver, the second most affected body system is the cardiovascular system which results in alcohol-induced pathologies such as atherosclerosis, hypertension, arrhythmias, and dilated cardiomyopathy. In newborns, prenatal alcohol exposure increases the risk of congenital heart disease and fetal alcohol spectrum disorder (FASD). Other drugs also have side effects of cardiac toxicity. Chemotherapy agents such as doxorubicin and anti-psychotic agents such as clozapine are several examples. Thus, there is a need to identify methods of reducing alcohol and other drug-induced cardiac toxicities.

Ganatra et al. report using dexrazoxane for the reduction of anthracycline-induced cardiotoxicity in adults with preexisting cardiomyopathy and cancer. Cardio-Oncology, 2019, 5:1.

Liu et al report chronic ethanol exposure induces deleterious changes in cardiomyocytes derived from human induced pluripotent stem cells. Stem Cell Rev Rep, 2021, 17(6): 2314-2331.

Forghani et al. report carfilzomib treatment causes molecular and functional alterations of human induced pluripotent stem cell-derived cardiomyocytes. J Am Heart Assoc, 2021, 10, e022247.

References cited herein are not an admission of prior art.

### SUMMARY

This disclosure relates to methods of reducing alcohol and other drug-induced cardiac toxicities by administering adrenalone, derivative, prodrug, ester, or salt thereof. In certain embodiments, this disclosure relates to methods of treating or preventing cardiotoxicity or a cardiac disease or condition by administering adrenalone, derivative, prodrug, ester, or salt thereof to a subject in need thereof. In certain embodiments, the subject is in need thereof due to administration of or consumption of alcohol or an anti-cancer agent that poses a risk of cardiotoxicity.

In certain embodiments, the subject is diagnosed with alcoholism. In certain embodiments, the subject is pregnant with a fetus, and the fetus is diagnosed with a heart defect. In certain embodiments, the subject is a newborn with prenatal alcohol exposure. In certain embodiments, the subject is diagnosed with fetal alcohol spectrum disorder (FASD) or a congenital heart disease.

In certain embodiments, the therapeutic agent that poses a risk of cardiotoxicity is a chemotherapy agent. In certain embodiments, the chemotherapy agent is an anthracycline. In certain embodiments, the anthracycline is doxorubicin, daunorubicin, idarubicin, epirubicin, or mitoxantrone. In certain embodiments, the chemotherapy agent is a proteasome inhibitor. In certain embodiments, the proteasome inhibitor is carfilzomib or bortezomib. In certain embodiments, the chemotherapy agent is a tyrosine kinase inhibitor. In certain embodiments, the tyrosine kinase inhibitor is sunitinib or nilotinib. In certain embodiments, the chemotherapy agent is cyclophosphamide. In certain embodiments, the chemotherapy agent is 5-fluorouracil.

In certain embodiments, the therapeutic agent that poses a risk of cardiotoxicity is clozapine.

In certain embodiments, the therapeutic agent that poses a risk of cardiotoxicity is cocaine, methamphetamine, or 3,4-methylenedioxymethamphetamine (MDMA).

In certain embodiments, the adrenalone, derivative, prodrug, ester, or salt thereof is administered in combination with dexrazoxane and/or folic acid.

### BRIEF DESCRIPTION OF THE SEVERAL VIEWS OF THE DRAWINGS

FIG. 1 shows data using adrenalone HCl (4H10) indicating protection of hiPSC-CMs from carfilzomib-induced cardiotoxicity. Cell viability was analyzed after IMR-90 hiPSC-CMs were treated with carfilzomib (CFZ) with or without 4H10 for 24 hours. (n=5 cultures).

FIG. 2 shows data indicating 4H10 mitigates carfilzomib-induced mitochondrial defects of hiPSC-CMs. MitoTracker™ Red staining was performed in IMR-90 hiPSC-CMs treated with CFZ with or without 4H10 for 24 hours.

FIG. 3 shows data indicating 4H10 mitigates carfilzomib-induced intracellular oxidative stress of hiPSC-CMs. The quantification of oxidative stress was performed using DCFDA staining of SCVI hiPSC-CMs treated with Cfz with or without 4H10 for 24 hours.

FIG. 4 shows data indicating 4H10 does not interfere with the proteasome inhibition activity of carfilzomib and the ability of carfilzomib to kill cancer cells. (top) Proteasome activity of IMR-90 hiPSC-CMs was measured after the cells were treated with CFZ with or without 4H10 for 24 hours. (bottom, right, left) Proteasome activity and cell viability of RPMI8226 cells (cancer cells) were measured after the cells were treated with CFZ with or without 4H10 for 24 hours.

FIG. 5 shows data indicating 4H10 protects hiPSC-CMs from doxorubicin-induced cytotoxicity.

### DETAILED DISCUSSION

Before the present disclosure is described in greater detail, it is to be understood that this disclosure is not limited to particular embodiments described, and as such may, of course, vary. An “embodiment” of this disclosure refers to an example and infers that the example is not necessarily limited to the example. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Adrenalone” is a compound with chemical name 1-(3,4-dihydroxyphenyl)-2-(methylamino) ethan-1-one, illustrated below:

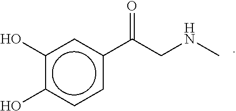

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other such that they are contained/circulating in the patient at the same time, e.g., considering half-lives.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. The derivative may be a prodrug. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, -NRaRb, -NRaC(═O)Rb, -NRaC(═O)NRaNRb, -NRaC(═O)ORb, -NRaSO2Rb, —C(═O)Ra, —C(═O)ORa, —C(═O)NRaRb, —OC(═O)NRaRb, -ORa, -SRa, -SORa, —S(═O)2Ra, —OS(═O)2Ra and —S(═O)2ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in compositions over the parent drug. A prodrug may be converted into the parent drug by various mechanisms, including enzymatic processes and metabolic hydrolysis. Typical prodrugs are esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol, i.e., hydroxy group.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

As used herein the term “isotonic” refers to a solution that has the same osmotic pressure as blood serum.

In certain embodiments, this disclosure relates to methods of treating or preventing cardiotoxicity by administering adrenalone, derivative, prodrug, ester, or salt thereof to a subject in need thereof. In certain embodiments, the subject is in need thereof due to administration of or consumption of alcohol, other addictive drug, or a therapeutic agent that poses a risk of cardiotoxicity.

In certain embodiments, the cardiotoxicity inducing drug and adrenalone, derivative, prodrug, ester, or salt thereof are administered at the same time or within a short period thereafter, e.g., with 5 minutes or 10 minutes. In certain embodiments, additional administrations of adrenalone, derivative, prodrug, ester, or salt thereof are administered one, two, or more times on schedule, e.g., hourly, every two hours, four hours, twelve hours, or daily.

In certain embodiments, the subject is diagnosed with alcoholism. In certain embodiments, the subject is pregnant with a fetus, and the fetus is diagnosed a heart defect. In certain embodiments, the subject is a newborn with prenatal alcohol exposure. In certain embodiments, the subject is diagnosed with fetal alcohol spectrum disorder (FASD) or a congenital heart disease.

In certain embodiments, the addictive drug that poses a risk of cardiotoxicity is cocaine, methamphetamine, or 3,4-methylenedioxymethamphetamine (MDMA).

In certain embodiments, the therapeutic agent that poses a risk of cardiotoxicity is a chemotherapy agent. In certain embodiments, the chemotherapy agent is an anthracycline. In certain embodiments, the anthracycline is doxorubicin, daunorubicin, idarubicin, epirubicin, or mitoxantrone.

In certain embodiments, the chemotherapy agent is a proteasome inhibitor. In certain embodiments, the proteasome inhibitor is carfilzomib or bortezomib. In certain embodiments, the chemotherapy agent is a tyrosine kinase inhibitor. In certain embodiments, the tyrosine kinase inhibitor is sunitinib or nilotinib. In certain embodiments, the chemotherapy agent is cyclophosphamide. In certain embodiments, the chemotherapy agent is 5-fluorouracil.

In certain embodiments, this disclosure relates to methods of treating or preventing a cardiovascular disease such as atherosclerosis, hypertension, arrhythmias, or dilated cardiomyopathy by administering adrenalone, derivative, prodrug, ester, or salt thereof to a subject in need thereof. In certain embodiments, the subject is in need thereof due to administration of or consumption of alcohol or a therapeutic agent that poses a risk of cardiotoxicity.

This disclosure relates to methods of treating or preventing a disease of the heart or vasculature by administering adrenalone, derivative, prodrug, ester, or salt thereof to a subject in need thereof. In certain embodiments, the subject experienced an abnormal cardiac event, or is exhibiting symptoms of, or diagnosed with coronary artery disease, angina, myocardial infarction, calcific aortic valve disease, stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, or venous thrombosis.

In certain embodiments, this disclosure relates to methods of counteracting cardiotoxicity during drug therapy for the treatment or prophylaxis of cancer. In certain embodiments, methods involve administering a formulation of adrenalone to a human patient. In certain embodiments, the formulation may be administered prior to the administration of an anticancer agents such as an anthracycline. In certain embodiments, adrenalone is in the form of a salt or a free base formulation. In certain embodiments, the pharmaceutical formulation is in the form of a tablet, capsule, pill, or micronized particle. In certain embodiments, adrenalone formulations described herein may be administered to a patient orally or intravenously. In certain embodiments, the adrenalone formulation is an isotonic solution which is suitable for intravenous administration to a patient in need of treatment.

In certain embodiments, adrenalone can be used in the manufacture of a medicament for treating or preventing cardiotoxicity, such as, cardiotoxicity associated with anthracycline administration, specifically doxorubicin administration or other anticancer agent. In certain embodiments, provide is the use of adrenalone and another agent, such as an anthracycline, for simultaneous separate or sequential administration. In certain embodiments, the agent is doxorubicin. In certain embodiments, the use is for treating cancer. In another embodiment, the use is for treating cancer without cardiotoxicity. In certain embodiments, the anticancer agent is abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, or zoledronic acid.

In certain embodiments, the cancer is leukemia or other hematological cancer. In certain embodiments, the cancer is a hematological malignancy such as acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), chronic myeloid leukemia (CML), myeloproliferative neoplasms (MPNs), and lymphomas, Hodgkin's lymphomas, and non-Hodgkin's lymphomas such as Burkitt lymphoma, B-cell lymphoma.

In certain embodiments, the cancer is a solid tumor, cellular malignancy, or hematological malignancy. In certain embodiments, the cancer is lung cancer, non-small cell lung cancer, small cell lung cancer, bronchus cancer, mesothelioma, malignant pleural mesothelioma, lung adenocarcinoma, breast cancer, prostate cancer, colon cancer, rectum cancer, colorectal cancer, gastrointestinal cancer, stomach cancer, esophageal cancer, ovarian cancer, cervical cancer, melanoma, kidney cancer, pancreatic cancer, pancreatic ductal adenocarcinoma (PDA), thyroid cancer, brain cancer, glioblastoma (GBM), medulloblastoma, glioma, neuroblastoma, liver cancer, bladder cancer, uterine cancer, bone cancer, osteosarcoma, sarcoma, rhabdomyosarcoma, Ewing's sarcoma, retinoblastoma, or nasopharyngeal carcinoma.

In the above-described methods of treatment and uses, adrenalone or derivative as disclosed herein may be employed alone, in combination with one or more other compounds disclosed herein or in combination with other therapeutic methods or agents. In certain embodiments, methods of treatment are done using adrenalone or derivative as disclosed herein, or a pharmaceutical composition comprising the same, in combination with administering a cardiotoxic inducing agent and optionally an additional supporting agent, a cardiac agent, or anti-inflammatory agent.

In certain embodiments, adrenalone, derivative, prodrug, ester, or salt thereof is administered in combination with a cardiac agent, ephedrine, atropine, a vasoconstrictor, a beta-adrenergic agent, isoproterenol, epinephrine, dobutamine, dopamine, norepinephrine, phenylephrine, norepinephrine, vasopressin, a phosphodiesterase inhibitor, amrinone, milrinone, a statin, atorvastatin, cerivastatin, fluvastatin, lovastatin, mevastatin, pitavastatin, pravastatin, rosuvastatin, simvastatin, ezetimibe, amlodipine, niacin, aspirin, omega-3 fatty acid, beta-blocker, calcium channel blocker, nifedipine, amlodipine, lacidipine, verapamil, diltiazem, or combinations thereof.

In certain embodiments, contemplated anti-inflammatory agents include alclofenac, alclometasone dipropionate, alpha amylase, amcinafal, amfenac sodium, anakinra, anirolac, balsalazide disodium, bendazac, benoxaprofen, bromelains, broperamole, budesonide, carprofen, cliprofen, clobetasol propionate, clobetasone butyrate, clopirac, cortodoxone, decanoate, deflazacort, depo-testosterone, desonide, desoximetasone, dexamethasone dipropionate, diclofenac potassium, diclofenac sodium, diflorasone diacetate, diflumidone sodium, diflunisal, difluprednate, dimethyl sulfoxide, enolicam sodium, etodolac, felbinac, fenamole, fenbufen, fenclofenac, fendosal, fenpipalone, fentiazac, flazalone, flufenamic acid, flunisolide acetate, flunixin, flunixin meglumine, fluorometholone acetate, flurbiprofen, fluticasone propionate, furaprofen, halcinonide, halobetasol propionate, ibuprofen, ibuprofen aluminum, ibuprofen piconol, indomethacin, indomethacin sodium, indoprofen, isoxepac, isoxicam, ketoprofen, lofemizole hydrochloride, loteprednol etabonate, meclofenamate sodium, meclofenamic acid, mefenamic acid, mesalamine, methenolone, methenolone acetate, nabumetone, nandrolone, naproxen, naproxen sodium, naproxol, olsalazine sodium, oxaprozin, oxyphenbutazone, oxymetholone, pirfenidone, piroxicam, piroxicam cinnamate, piroxicam olamine, pirprofen, proquazone, proxazole, proxazole citrate, salsalate, stanozolol, sudoxicam, sulindac, suprofen, talniflumate, tenidap, tenidap sodium, tenoxicam, testosterone, testosterone blends, tiopinac, tixocortol pivalate, tolmetin, tolmetin sodium, triclonide, triflumidate, zidometacin, zomepirac, or combinations thereof.

In certain embodiments, this disclosure relates to methods of counteracting cardiotoxicity during clozapine therapy for the treatment or prophylaxis of a psychiatric disorder. In certain embodiments, this disclosure relates to method of treating psychiatric disorders by administering an effective amount adrenalone in combination with clozapine to a subject in need thereof to counteract cardiotoxicity. In certain embodiments, the psychiatric disorder is schizophrenia and/or to reduce the risk of suicidal behavior.

In certain embodiments, the adrenalone, derivative, prodrug, ester, or salt thereof is administered in combination with dexrazoxane and/or folic acid.

The precise therapeutically effective amount of an adrenalone or derivative as disclosed herein will depend on a number of factors. These variables determine what dose of compound needs to be administered in a sufficient percentage and for a sufficient amount of time to have the desired effect on the condition being treated. The amount of compound administered will also depend on factors related to patients and disease including, but not limited to, the following: the age, weight, concomitant medications, and medical condition of the subject being treated, the precise condition requiring treatment and its severity, the nature of the formulation, and the route of administration. Ultimately, the dose will be at the discretion of the attendant physician or veterinarian. Typically, the compound disclosed herein will be given for treatment in the range of 0.01 to 30 mg/kg body weight of recipient (mammal) per day or per dose or per cycle of treatment and more usually in the range of 0.1 to 10 mg/kg body weight per day or per dose or per cycle of treatment. Thus, for an adult human being treated for a condition, the actual amount per day or per dose or per cycle of treatment would usually be from 1 to 2000 mg and this amount may be given in a single or multiple doses per day or per dose or per cycle of treatment. The full spectrum of dosing regimens may be employed ranging from continuous dosing (with daily doses) to intermittent dosing.

Chronic Ethanol Exposure Induces Deleterious Changes in Cardiomyocytes Derived from Human Induced Pluripotent Stem Cells

Cardiomyocytes derived from human pluripotent stem cells (hiPSC-CMs) were treated with clinically relevant doses of ethanol for various durations up to 5 weeks. The treated cells were characterized for their cellular properties and functions, and global proteomic profiling was conducted. Increased cell death, oxidative stress, deranged Ca²⁺ handling, abnormal action potential, altered contractility, and suppressed structure development were observed in ethanol-treated cells. Many dysregulated proteins identified by global proteomic profiling were involved in apoptosis, heart contraction, and extracellular collagen matrix. In addition, several signaling pathways including the Wnt and TGFβ signaling pathways were affected due to long-term ethanol exposure. This data indicates chronic ethanol exposure to hiPSC-CMs induces cardiotoxicity, impairs cardiac functions, and alters protein expression and signaling pathways. These results indicate the utility of hiPSC-CMs as a model for chronic alcohol exposure.

Culture of hiPSCs and Cardiomyocyte Differentiation

A human induced pluripotent cell line (hiPSC line IMR90™, WiCell Research Institute) was fed daily on Matrigel™-coated plates with a defined medium for hiPSC culture, mTeSR1 medium. For CM differentiation, hiPSCs were induced using a growth factor-driven differentiation protocol as reported in Jha et al. Efficient differentiation of cardiomyocytes from human pluripotent stem cells with growth factors. Methods Mol Biol, 2015, 1299, 115-131. On the day of induction (day 0), medium was replaced with RPMI 1640 medium supplemented with 2% B27 minus insulin (a cell culture supplement) containing 100 ng/ml activin A. After induction for 24 h, the medium was replaced with fresh RPMI supplemented with 2% B27 minus insulin containing 10 ng/ml BMP4. From day 4 of differentiation, the medium was replaced with RPMI supplemented with 2% B27. On day 5, the cells were dissociated and reseeded into AggreWell400™ plates to acquire cardiac spheres. After 24 h, the cardiac spheres formed were collected and transferred to low-adhesion dishes for suspension culture. The medium was changed every other day. Cardiac spheres typically started beating spontaneously by day 7 to 9.

Screening Molecules for their Cardioprotective Capacity to Mitigate Chemotherapy-Induced Cardiotoxicity

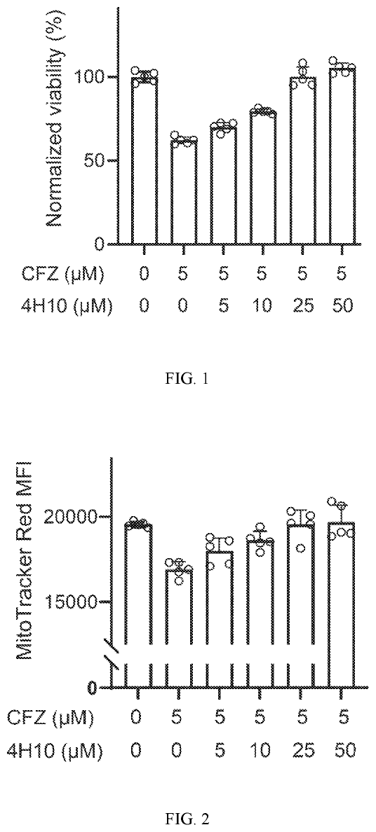

Cardiomyocytes derived from human pluripotent stem cells (hiPSC-CMs) were provide a new platform for the studies of cancer drug-induced side effects and therapies. Using these cells, lead molecules were identified indicating protective properties on cardiomyocytes from the toxic effect of chemotherapy agents and ethanol (EtOH). Treatment of hiPSC-CMs with carfilzomib, a drug for the treatment of multiple myeloma, caused cytotoxicity, consistent with cardiac adverse events in patients on therapy. Among the proteosome inhibitors, carfilzomib has the highest cardiotoxicity in clinic. When the cells were co-treated with carfilzomib and the small molecule adrenalone HCl (4H10), reduced cytotoxicity was observed (FIG. 1). Adrenalone also reduced mitochondrial oxidative stress caused by bortezomib (PS-341), another proteosome inhibitor for the treatment of multiple myeloma. Oxidative stress is a common molecular mechanism for cardiotoxicity induced by chemotherapy including proteosome inhibitors.

Experiments indicate that adrenalone HCl can protect hiPSC-CMs from alcohol- and carfilzomib-induced cytotoxicity. Adrenalone HCl increased cell survival dose-dependently following the challenge of hiPSC-CMs with EtOH as determined by a cell viability assay and cell counting using high-content imaging. In addition, adrenalone HCl reduced oxidative stress and defects in the expression of cardiac structural protein cardiac troponin T and mitochondrial content in EtOH-treated hiPSC-CMs. EtOH-induced cardiotoxicity shares common disease phenotypes and possibly similar underlying mechanisms with drug-induced cardiotoxicity. Adrenalone HCl also protected hiPSC-CMs from cardiotoxicity induced by carfilzomib (a chemotherapeutic drug). Adrenalone HCl also increased cell viability in EtOH-treated hiPSC-CMs derived from another cell line SCVI-273 hiPSCs. Adrenalone HCl attenuated the reduced cell number induced by EtOH treatment as detected by high-content imaging.

Treatment of hiPSC-CMs with carfilzomib negatively affects contractility, Ca²⁺ handling, traction forces, and mitochondrial function. Treatment of hiPSC-CMs with chemotherapeutic agent melphalan induces cardiotoxicity, including oxidative stress and abnormal cardiac function. These results are consistent with clinically observed arrhythmias in patients receiving carfilzomib and melphalan.

4H10 Protects hiPSC-CMs from Carfilzomib-Induced Cytotoxicity

Carfilzomib has demonstrated efficacy in the treatment of multiple myeloma, but it can cause cardiotoxicity including heart failure and cardiac arrhythmias in cancer patients. Consistently, treatment of hiPSC-CMs induced cell death and cytotoxicity. To examine if 4H10 could protect cardiomyocytes from carfilzomib-induced cytotoxicity, hiPSC-CMs were treated with carfilzomib at 5 μM alone or together with 4H10 at various concentrations for 24 h and then examined cell viability using the CellTiter-Blue Cell Viability Assay. Cells treated with DMSO (vehicle) were used as a control. The dose of carfilzomib was selected based on the maximum (or peak) serum concentration (Cmax of 5.88 μM) and findings on the cytotoxicity effect of carfilzomib on hiPSC-CMs. A significant decrease in cell viability was detected in carfilzomib-treated cells compared with DMSO-treated cells (FIG. 1). In contrast, the cells co-treated with carfilzomib and 4H10 at concentrations of 5 μM or higher had significantly increased cell viability compared with the cells treated with DMSO. 4H10 at 25 μM and 50 μM provided the maximal cytoprotective effect for the cells to reach viability levels similar to those from DMSO-treated cells (FIG. 1). These results demonstrated that 4H10 mitigated carfilzomib-induced cytotoxicity in a dose-dependent manner.

4H10 Mitigates Carfilzomib-Induced Mitochondrial Defects of hiPSC-CMs

Mitochondria play a crucial role in energy production, cellular metabolism, and the regulation of cell death. Experiments indicate carfilzomib induces mitochondrial dysfunction in hiPSC-CMs. To assess the potential mitigating effects of 4H10 on carfilzomib-induced mitochondrial dysfunction, hiPSC-CMs were treated with carfilzomib alone or co-treated with 4H10 at various concentrations. Mitochondria were then visualized using MitoTracker™ Red dyes, which can accumulate in active mitochondria and allow for the measurement of mitochondrial content and activity in live cells. Carfilzomib treatment led to a reduction in mitochondrial mass and activity, as indicated by the decreased fluorescence intensity of MitoTracker™ Red as analyzed by high-content imaging. In contrast, co-treatment with 4H10 at 10 μM or higher concentrations resulted in an increase in fluorescence intensity, and this effect was 4H10 dose-dependent (FIG. 2). These results indicated that 4H10 mitigated carfilzomib-induced mitochondrial defects in hiPSC-CMs in a dose-dependent manner.

4H10 Mitigates Carfilzomib-Induced Oxidative Stress of hiPSC-CMs

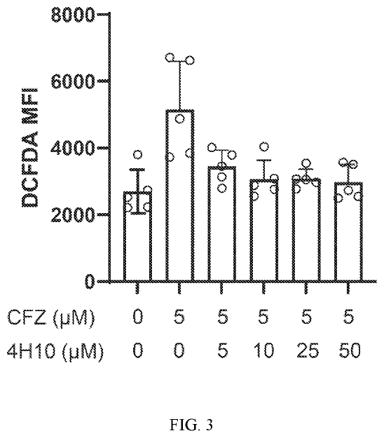

The induction of oxidative stress is a crucial mechanism through which proteasome inhibitors induce apoptosis. To investigate if 4H10 could alleviate carfilzomib-induced increase of oxidative stress in cardiomyocytes, hiPSC-CMs were exposed to carfilzomib at 5 μM alone or together with 4H10 at various concentrations for 24 h. The production of cellular reactive oxygen species (ROS) was then measured using DCFDA/H2DCFDA assay kit. Carfilzomib treatment led to a significant increase in DCFDA fluorescence intensity, indicating an increased oxidative stress generation. In contrast, co-treatment with 4H10 at 5 μM or higher concentrations resulted in a reduction of DCFDA fluorescence intensity, and this effect was 4H10 dose-dependent (FIG. 3). These findings indicated that 4H10 mitigated carfilzomib-induced oxidative stress in hiPSC-CMs in a dose-dependent manner.

4H10 does not Interfere with the Proteasome Inhibition Activity and Anticancer Efficacy of Carfilzomib

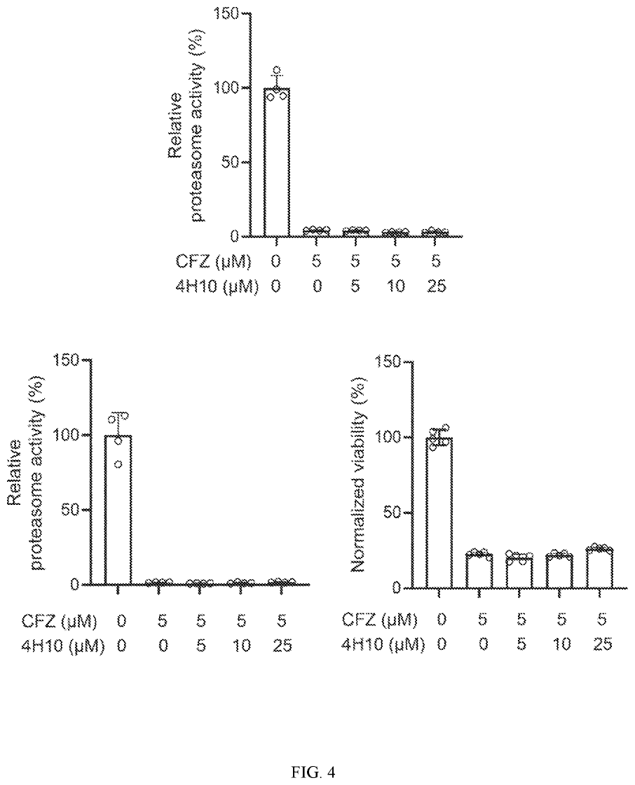

The proteasome, responsible for the regulated degradation of proteins, is a key cellular component targeted by carfilzomib as part of its mechanism of action in cancer therapy. Therefore, in addition to validation of cardioprotective effects of 4H10 on cell viability and mitochondrial function, it is crucial to examine if 4H10 compromises the proteasome inhibition efficacy of carfilzomib. Following carfilzomib treatment in hiPSC-CMs, a significant decrease in cellular proteasome activity was observed when compared with cells treated with DMSO (FIG. 4). However, 4H10 co-treatment did not interfere with the proteasome inhibition activity of carfilzomib (FIG. 4).

Experiments were designed to determine the effects of carfilzomib and 4H10 on proteasome activity and cell viability of RPMI8226 cell line, which has been investigated for the efficacy tests of carfilzomib in lymphoma. Proteasome activity in RPMI8226 cells was suppressed by carfilzomib treatment compared to DMSO-treated cells, while 4H10 did not elicit any changes. The cell viability assay demonstrated that carfilzomib significantly decreased the viability of RPMI8226 cells compared to DMSO-treated cells, which was not significantly changed by 4H10 co-treatment. These results suggested a unique property of 4H10 in selectively mitigating carfilzomib-induced toxicity in cardiomyocytes without compromising ability of carfilzomib to inhibit proteasome activity and induce an anticancer efficacy.

hiPSC-CMs Express α1-Adrenergic Receptor (α1-AR)

The α1-AR is known to be a potential target of 4H10. To assess if the hiPSC-CMs express α1-AR, the cells were stained using fluorescent BODIPY-FL-prazosin, which is a high-affinity antagonist for the α1-AR and can be used to detect the expression and subcellular localization of α1-AR. The expression of α1-AR was detected in hiPSC-CMs based on the BODIPY FL prazosin signals. In addition, the BODIPY FL prazosin signals were co-localized with nuclei. These results are consistent with the nuclear localization of α1-AR in adult mouse cardiac myocytes.

4H10 Protects hiPSC-CMs from Doxorubicin-Induced Cytotoxicity

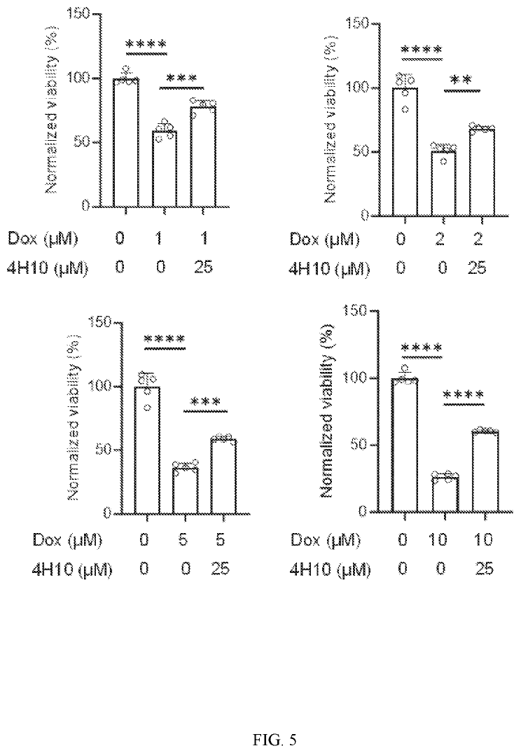

Doxorubicin is a widely used chemotherapy drug for various cancers, including breast cancer, leukemia, and lymphoma. However, one significant side effect associated with doxorubicin use is cardiotoxicity. It is contemplated that 4H10 may target common mechanisms of cardiotoxicity and have a broad impact of cardioprotection since it protects hiPSC-CMs from toxicity induced by both ethanol and carfilzomib. To evaluate if 4H10 could also protect cardiomyocytes from doxorubicin-induced cytotoxicity, hiPSC-CMs were treated with doxorubicin at 1, 2, 5 or 10 μM alone (the Cmax of doxorubicin is 1.7 μM13), or together with 4H10 at 25 μM for 24 h and cell viability was examined using the CellTiter-Blue™ Cell Viability Assay. A significant decrease in cell viability in cells treated with doxorubicin was detected at 1-10 μM alone compared with untreated cells (FIG. 5). In contrast, the cells co-treated with doxorubicin and 4H10 showed increased cell viability compared with the cells treated with doxorubicin (FIG. 5). These results indicated that 4H10 also mitigated doxorubicin-induced cytotoxicity.

1. A method of treating or preventing cardiotoxicity comprising administering adrenalone or salt thereof to a subject in need thereof.

2. The method of claim 1, wherein the subject is in need thereof due to administration of or consumption of alcohol or a therapeutic agent that poses a risk of cardiotoxicity.

3. The method of claim 2, the subject is diagnosed with alcoholism.

4. The method of claim 2, the subject is pregnant with a fetus, and the fetus is diagnosed a heart defect.

5. The method of claim 1, wherein the subject is a newborn with prenatal alcohol exposure.

6. The method of claim 5, wherein the subject is diagnosed with fetal alcohol spectrum disorder (FASD) or a congenital heart disease.

7. The method of claim 2, wherein the therapeutic agent that poses a risk of cardiotoxicity is a chemotherapy agent.

8. The method of claim 7, wherein the chemotherapy agent is an anthracycline.

9. The method of claim 8, wherein the anthracycline is doxorubicin, daunorubicin, idarubicin, epirubicin, or mitoxantrone.

10. The method of claim 7, wherein the chemotherapy agent is a proteasome inhibitor.

11. The method of claim 10, wherein the proteasome inhibitor is carfilzomib or bortezomib.

12. The method of claim 7, wherein the chemotherapy agent is a tyrosine kinase inhibitor.

13. The method of claim 12, wherein the tyrosine kinase inhibitor is sunitinib or nilotinib.

14. The method of claim 7, wherein the chemotherapy agent is cyclophosphamide.

15. The method of claim 7, wherein the chemotherapy agent is 5-fluorouracil.

16. The method of claim 2, wherein the therapeutic agent that poses a risk of cardiotoxicity is clozapine.

17. The method of claim 2, wherein the therapeutic agent that poses a risk of cardiotoxicity is cocaine, methamphetamine, or 3,4-methylenedioxymethamphetamine (MDMA).

18. The method of claim 1, wherein adrenalone or salt thereof is administered in combination with dexrazoxane.

19. A method of treating a cardiovascular disease comprising administering an effective amount of adrenalone to a subject in need thereof.
